(19) United States
(12) Patent Application Publication
(10) Pub. No.: US 2025/0073054 A1
(43) Pub. Date: Mar. 6, 2025

(54) EXTERNAL NASAL DILATOR

(71) Applicant: ASO LLC, Sarasota, FL (US)

(72) Inventors: Edmund A. Sinda, Bradenton, FL (US); Brooke A. Hagerty, Bradenton, FL (US); Kyoko Asami, Sarasota, FL (US); Jignesh Barot, Bradenton, FL (US); Janet Luong, Cincinnati, OH (US); Kelly Lee Schmeichel, Cincinnati, OH (US); Jacek Tadeusz Krauzowicz, Cincinnati, OH (US)

(21) Appl. No.: 18/825,557

(22) Filed: Sep. 5, 2024

Related U.S. Application Data
(60) Provisional application No. 63/536,791, filed on Sep. 6, 2023.

(51) Int. Cl. A61F 5/08

(52) U.S. Cl. CPC A61F 5/08

(57) ABSTRACT

An external nasal dilator includes a flexible substrate having a top edge, bottom edge, a left edge, and a right edge that collectively define an outer perimeter of the external nasal dilator. A left recess is along the left edge and a right recess is along the right edge. The left recess and right recess divide the flexible substrate into two laterally opposed upper lobes and two laterally opposed lower lobes. An upper section including the two laterally opposed upper lobes carries an upper resilient member upper resilient member that applies a first dilating force to the internal nasal valves. A lower section including the two laterally opposed lower lobes carries a lower resilient member that is distinct from the upper resilient member and applies a second dilating force to the external nasal valves. An intermediate section longitudinally separates the upper resilient member and the lower resilient member.

### CROSS-REFERENCE TO RELATED APPLICATION

This claims the benefit of priority from U.S. Application No. 63/536,791, filed Sep. 6, 2024, which is incorporated by reference in its entirety.

### FIELD

This relates to the field of nasal dilators and, more particularly, to external nasal dilators.

### BACKGROUND

External nasal dilators help with breathing through the nose. They gently expand the nasal passages by applying an outward dilating force that supports the nasal passages from collapsing inwardly when the wearer inhales. Such dilators are composed of one or more resilient band of material attached to an adhesive substrate. When the dilator is adhered to the skin of the nose the resilient material acts as a spring, applying an outward force to the nose during inhalation.

### BRIEF SUMMARY

A problem with conventional commercially available external nasal dilators is that they target the internal nasal valves only, essentially ignoring the external nasal valves. An improved external nasal dilator that applies dilating force to both the internal and external nasal valves is needed. It would also be advantageous to separate the dilating force applied to the internal nasal valves from the dilating force applied to the external nasal valves and to provide additional adhesive surface area on the portion of the external nasal dilator that targets the external nasal valves. The new external nasal dilators described herein solve one or more of these problems.

A first example of such an external nasal dilator is configured to dilate laterally opposed internal nasal valves and external nasal valves of a human nose. The external nasal dilator comprises a flexible substrate having an adhesive back surface for adhering the external nasal dilator to a human nose. The flexible substrate has a top edge, bottom edge, a left edge, and a right edge that collectively define an outer perimeter of the external nasal dilator. The left edge and the right edge converge inwardly toward each other to form a left recess along the left edge and a right recess along the right edge. The left recess and right recess divide the flexible substrate into two laterally opposed upper lobes and two laterally opposed lower lobes. An upper section of the flexible substrate includes the two laterally opposed upper lobes and carries an upper resilient member extending laterally from one upper lobe to the other upper lobe. When the external nasal dilator is adhered to the nose, the upper resilient member applies a first dilating force to the internal nasal valves. A lower section of the flexible substrate includes the two laterally opposed lower lobes and carries a lower resilient member that is distinct from the upper resilient member and extends laterally from one lower lobe to the other lower lobe. The lower resilient member and the lower section being laterally elongated relative to the upper resilient member and the upper section, respectively. When the external nasal dilator is adhered to the nose, the lower resilient member applies a second dilating force to the external nasal valves. An intermediate section of the flexible substrate extends laterally between, and longitudinally separates, the upper resilient member and the lower resilient member. The left recess and the right recess are in the intermediate section.

The external nasal dilator may also include one or more of the additional features discussed below.

The intermediate section, right recess, and left recess may separate the first dilation force at the upper lobes from the second dilation force at the lower lobes.

The adhesive back surface may have greater surface area on the lower section than the upper section.

The second dilation force may be greater than the first dilation force.

A left convergence point defining an innermost edge of the left recess and a right convergence point defining an innermost edge of the right recess may be closer to the upper resilient member than the lower resilient member.

A plurality of discrete slits may be through the intermediate section, the discrete slits being laterally spaced apart and longitudinally elongated.

A pair of opposed cuts may extend longitudinally through the flexible substrate, respectively, at laterally opposed ends of the lower resilient member. The opposed cuts define a pair of opposed flaps of the lower resilient member that direct the second dilating force. The upper resilient member has a lateral length from a left end thereof to a right end thereof and a distance between the opposed cuts is less than the lateral length.

A second example of such an external nasal dilator is configured to dilate laterally opposed internal nasal valves and external nasal valves of a human nose. The external nasal dilator comprises a flexible substrate having an adhesive back surface for adhering the external nasal dilator to a human nose. The flexible substrate has a top edge, bottom edge, a left edge, and a right edge that collectively define an outer perimeter of the external nasal dilator. An upper section of the flexible substrate carries an upper resilient member extending laterally across the upper section. When the external nasal dilator is adhered to the nose, the upper resilient member applies a first dilating force to the internal nasal valves. A lower section of the flexible substrate carries a lower resilient member that is distinct from the upper resilient member and extends laterally from across the lower section. When the external nasal dilator is adhered to the nose, the lower resilient member applies a second dilating force to the external nasal valves. An intermediate section of the flexible substrate extends laterally between, and longitudinally separates, the upper resilient member and the lower resilient member. A plurality of discrete slits through the intermediate section are laterally spaced apart and longitudinally elongated.

The external nasal dilator may also include one or more of the additional features discussed below.

The left edge and the right edge may converge inwardly toward each other to form a left recess along the left edge and a right recess along the right edge. The left recess and right recess divide the flexible substrate into two laterally opposed upper lobes and two laterally opposed lower lobes. The upper section includes the two laterally opposed upper lobes and the upper resilient member extends laterally from one upper lobe to the other upper lobe. The lower section includes the two laterally opposed lower lobes. The lower resilient member extends laterally from one lower lobe to the other lower lobe. The lower resilient member and the lower section are laterally elongated relative to the upper resilient member and the upper section, respectively. The left recess and the right recess are in the intermediate section.

The slits may be longitudinally elongated towards a longitudinal midline of the external nasal dilator.

The slits may be longitudinally elongated towards a longitudinal midline of the external nasal dilator at an angle of 15° to 75° relative to the lateral direction.

The left edge and the right edge may converge inwardly toward each other to form a left recess along the left edge and a right recess along the right edge. The left recess and right recess divide the flexible substrate into two laterally opposed upper lobes and two laterally opposed lower lobes. The upper section includes the two laterally opposed upper lobes. The upper resilient member extends laterally from one upper lobe to the other upper lobe. The lower section includes the two laterally opposed lower lobes. The lower resilient member extends laterally from one lower lobe to the other lower lobe. The left recess and the right recess are in the intermediate section. The intermediate section, right recess, and left recess separate the first dilation force at the upper lobes from the second dilation force at the lower lobes.

The adhesive back surface may have greater surface area on the lower section than the upper section.

The lower resilient member and the lower section may be laterally elongated relative to the upper resilient member and the upper section.

The adhesive back surface may have greater surface area on the lower section than the upper section. The lower resilient member and the lower section are laterally elongated relative to the upper resilient member and the upper section. The second dilation force is greater than the first dilation force.

The left edge and the right edge may converge inwardly toward each other to form a left recess along the left edge and a right recess along the right edge, the left recess and right recess dividing the flexible substrate into two laterally opposed upper lobes and two laterally opposed lower lobes. The upper section includes the two laterally opposed upper lobes. The upper resilient member extends laterally from one upper lobe to the other upper lobe. The lower section includes the two laterally opposed lower lobes. The lower resilient member extends laterally from one lower lobe to the other lower lobe. The left recess and the right recess are in the intermediate section. A left convergence point defining an innermost edge of the left recess and a right convergence point defining an innermost edge of the right recess are closer to the upper resilient member than the lower resilient member.

A pair of opposed cuts may extend longitudinally through the flexible substrate at respective laterally opposed ends of the lower resilient member. The opposed cuts define a pair of opposed flaps of the lower resilient member that direct the second dilating force. The upper resilient member has a lateral length from a left terminal end thereof to a right terminal end thereof. A distance between the opposed cuts is less than the lateral length.

A pair of opposed cuts may extend longitudinally through the flexible substrate, respectively, at laterally opposed ends of the lower resilient member. The opposed cuts define a pair of opposed flaps of the lower resilient member that direct the second dilating force. The upper resilient member has a lateral length from a left terminal end thereof to a right terminal end thereof and a distance between the opposed cuts is less than the lateral length.

A third example of such an external nasal dilator is configured to dilate laterally opposed internal nasal valves and external nasal valves of a human nose. The external nasal dilator comprises a flexible substrate having an adhesive back surface for adhering the external nasal dilator to a human nose. The flexible substrate has a top edge, bottom edge, a left edge, and a right edge that collectively define an outer perimeter of the external nasal dilator. The left edge and the right edge converge inwardly toward each other to form a left recess along the left edge and a right recess along the right edge. The left recess and right recess divide the flexible substrate into two laterally opposed upper lobes and two laterally opposed lower lobes. An upper section of the flexible substrate includes the two laterally opposed upper lobes and carries an upper resilient member extending laterally from one upper lobe to the other upper lobe. When the external nasal dilator is adhered to the nose, the upper resilient member applies a first dilating force to the internal nasal valves. A lower section of the flexible substrate includes the two laterally opposed lower lobes and carries a lower resilient member that is distinct from the upper resilient member and extends laterally from one lower lobe to the other lower lobe. When the external nasal dilator is adhered to the nose, the lower resilient member applies a second dilating force to the external nasal valves. An intermediate section of the flexible substrate extends laterally between, and longitudinally separates, the upper resilient member and the lower resilient member. The left recess and the right recess are in the intermediate section. A left convergence point defines an innermost edge of the left recess and a right convergence point defines an innermost edge of the right recess. The left convergence point and the right convergence point are closer to the upper resilient member than the lower resilient member.

The external nasal dilator may also include one or more of the additional features discussed below.

The intermediate section, right recess, and left recess may separate the first dilation force at the upper lobes from the second dilation force at the lower lobes.

The adhesive back surface may have greater surface area on the lower section than the upper section.

The second dilation force may be greater than the first dilation force.

A plurality of discrete slits may be through the intermediate section, the discrete slits being laterally spaced apart and longitudinally elongated.

A pair of opposed cuts may extend longitudinally through the flexible substrate, respectively, at laterally opposed ends of the lower resilient member. The opposed cuts define a pair of opposed flaps of the lower resilient member that direct the second dilating force. The upper resilient member has a lateral length from a left end thereof to a right end thereof and a distance between the opposed cuts is less than the lateral length.

Each of these examples of the external nasal dilator may also include any of the features of the other examples.

### BRIEF DESCRIPTION OF THE DRAWINGS

FIG. 1 is a partial cutaway view of a left side of a human nose.

FIG. 2 is a bottom view of FIG. 1.

FIG. 3 is a front view of a first example of an external nasal dilator.

FIG. 4 is a back view of the external nasal dilator of FIG. 1.

FIG. 5 is a front left perspective view of the external nasal dilator of FIG. 1.

FIG. 6 is a back view of the external nasal dilator of FIG. 1, including a release liner.

FIG. 7 is a front perspective exploded view of the external nasal dilator of FIG. 1.

FIG. 8 is a top view of the external nasal dilator of FIG. 1.

FIG. 9 is a bottom view of the external nasal dilator of FIG. 1.

FIG. 10 is a left side view of the external nasal dilator of FIG. 1.

FIG. 11 is a right side view of the external nasal dilator of FIG. 1.

FIG. 12 is a left perspective view of the external nasal dilator of FIG. 1 adhered to a human nose.

FIG. 13 is a left perspective view of the external nasal dilator of FIG. 1 adhered to a human nose, showing the external and internal nasal valves.

FIG. 14 is a bottom view of FIG. 13.

FIG. 15 is a front view of a second example of the external nasal dilator.

FIG. 16 is a back view of the external nasal dilator of FIG. 15.

FIG. 17 is an enlarged view of FIG. 15.

FIG. 18 is a front view of the nasal dilator of FIG. 3 (upper panel) and a front view of the nasal dilator of FIG. 15 (lower panel) with letters and arrows representing measurements between different points on each dilator.

### DETAILED DESCRIPTION OF EXAMPLES

This describes certain examples, but not all possible examples of the external nasal dilator and related methods. Where a particular feature is disclosed in the context of a particular example, that feature can also be used, to the extent possible, in combination with and/or in the context of other examples. The external nasal dilator and related methods may be embodied in many different forms and should not be construed as limited to only the features and examples described here.

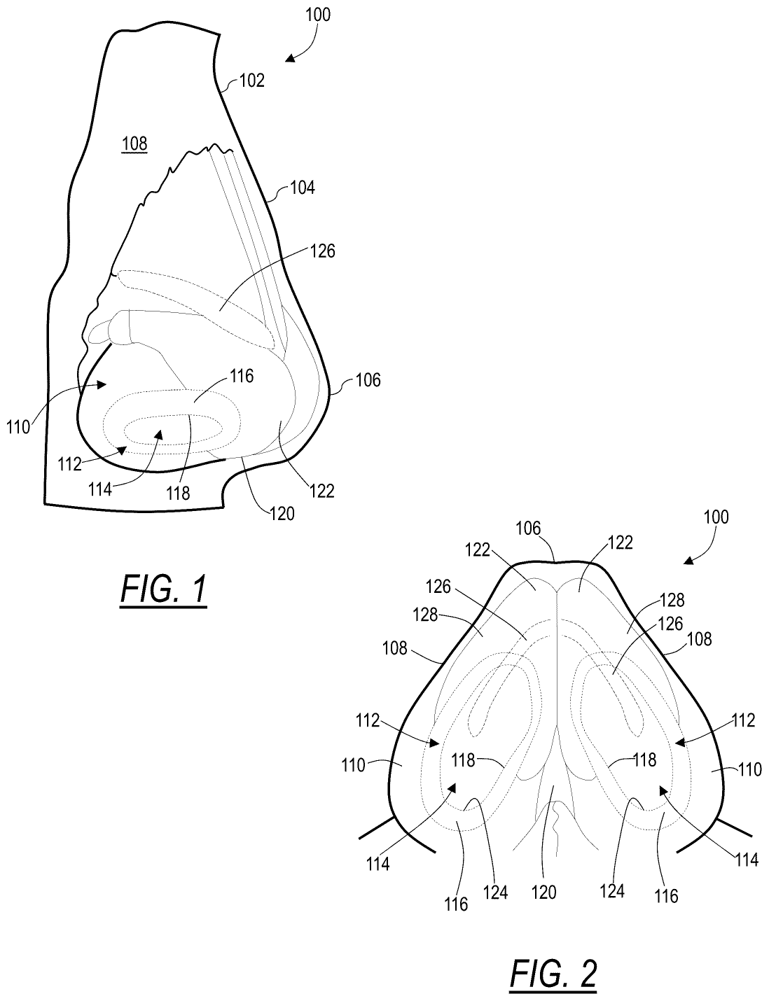

Because the external nasal dilator is described below with respect to parts of a human nose, aspects the human nose are first discussed by referring to FIGS. 1 and 2. In FIGS. 1 and 2, portions of the nose that are under the skin are shown in light lines and broken lines. The exterior of the nose 100 includes a root 102, bridge 104, tip 106, exterior wall 108, ala 110, and nostrils 112. The nostrils 112 define an opening to nasal cavities 114 or air passages.

Each nostril 112 further defines an external nasal valve 116, which is bounded by the ala 110, alar rim 118, septum 120, medial crura 122, and nasal sill 124. The external nasal valves 116 are shown in short dash lines.

Further inside each nasal cavity 114 is an internal nasal valve 126, which is bounded by the septum 120, head of the inferior turbinate, and the upper lateral cartilage 128. The internal nasal valves 126 are shown in long dash broken lines. Physiological conditions that narrow either the external nasal valves 116 or internal nasal valves 126 can restrict airflow through the nasal cavities 114.

When properly positioned over the bridge 104 of the nose 100, an external nasal dilator exerts a recoil, or dilating, force on the exterior wall 108 over the nasal cavities 114. This dilating force increases the cross-sectional area of the nasal cavities 114 at the level of the internal nasal valves 126 and external nasal valves 116.

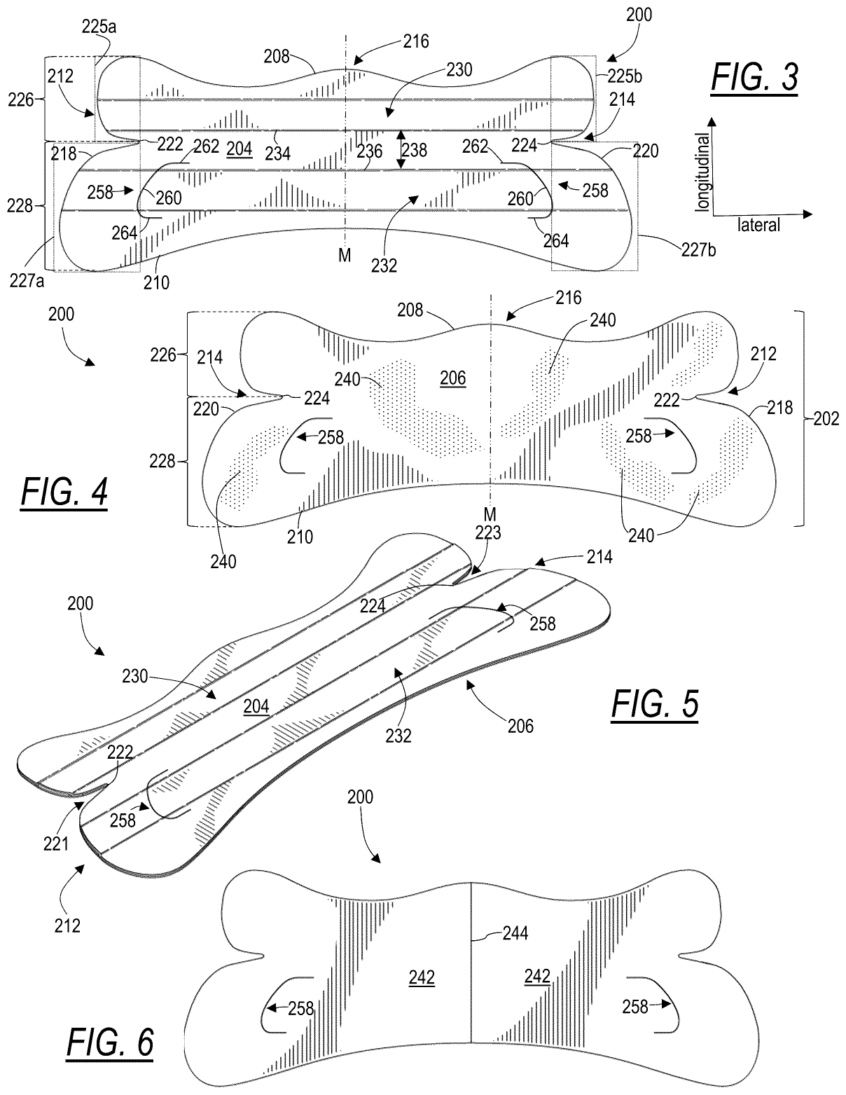

Referring to FIGS. 3-5, a first example of the external nasal dilator 200 includes a front surface 204, back surface 206 opposite the front surface 204, top edge 208, bottom edge 210 opposite the top edge 208, left end 212, and right end 214 opposite the left end 212. The external nasal dilator 200 extends in a lateral direction from the left end 212 to the right end 214 and longitudinally from the top edge 208 to the bottom edge 210. The external nasal dilator 200 is substantially symmetric about an imaginary longitudinal midline M.

The top edge 208 defines the top perimeter of the external nasal dilator 200 and extends from the left end 212 to the right end 214. A central region of the top edge 208 includes a nose placement member 216, forming a symmetric protrusion along the top edge 208 for helping a wearer visualize where to place the external nasal dilator 200 on the nose. In use, the wearer will place the nose placement member 216 on the bridge 104 evenly about the midline M. When worn properly, the midline M is substantially coaxial with an anatomical midline of the nose 100. The nose placement member 216 assists with proper placement on the nose to permit the external nasal dilator 200 to dilate the internal and external nasal valves.

The top edge 208 has a concave curvature between the midline M and left end 212 and the midline M and right end 214 such that the top edge 208 curves downwardly from the respective left end 212 and right end 214 then upwardly approaching the midline M.

The bottom edge 210 defines a bottom perimeter of the external nasal dilator 200 and extends from the left end 212 to the right end 214. The left end 212 defines a left edge 218 and the right end 214 defines a right edge 220. Between the left end 212 and right end 214, the bottom edge 210 has a concave curvature such that the bottom edge 210 curves upwardly approaching the midline M from the left end 212 and right end 214 respectively. This concave curvature is advantageous because it allows for a lower section 228 to extend downwardly toward the external nasal valves 116 when worn, which provides greater adhesive surface area located in close proximity to the external nasal valves 116 and less adhesive surface area located near the bridge 104.

The left edge 218 defines the left perimeter of the external nasal dilator 200. The right edge 220 defines a right perimeter of the external nasal dilator 200. The left edge 218 and right edge 220 join with the top edge 208 and bottom edge 210 to collectively define the outer perimeter of the external nasal dilator 200.

The left edge 218 and the right edge 220 converge inwardly toward each other to form a left recess 221 along the left edge 218 and a right recess 223 along the right edge 220. The left recess 221 and right recess 223 divide the external nasal dilator 200 into two laterally opposed upper lobes 225a,b and two laterally opposed lower lobes 227a,b.

In the left recess 221 and the right recess 223, the left edge 218 and right edge 220 converge inwardly toward the midline M to a left convergence point 222 and a right convergence point 224, respectively.

The region above the left convergence point 222 and right convergence point 224 to the top edge 208 is an upper section 226 of the external nasal dilator 200. The region below the left convergence point 222 and right convergence point 224 to the bottom edge 210 is the lower section 228 of the external nasal dilator 200.

The upper section 226 includes the two laterally opposed upper lobes 225a,b and carries an upper resilient member 230 extending laterally from one upper lobe 225a to the other upper lobe 225b. When the external nasal dilator 200 is adhered to the nose, the upper resilient member 230 applies a first dilating force to the internal nasal valves.

The upper resilient member 230 is positioned on the external nasal dilator 200 in such a way that the upper resilient member 230 extends laterally across the upper section 226 from the left edge 218 to the right edge 220 and applies the first dilating force to the laterally opposed internal nasal valves 126 when the external nasal dilator 200 is attached to the human nose.

The lower section 228 includes the two laterally opposed lower lobes 227a,b and carries a lower resilient member 232 that is distinct from the upper resilient member 230 and extends laterally from one lower lobe 227a to the other lower lobe 227b. The lower resilient member 232 and the lower section 228 are laterally elongated relative to the upper resilient member 230 and the upper section 226, respectively. When the external nasal dilator 200 is adhered to the nose, the lower resilient member 232 applies a second dilating force to the external nasal valves.

The lower resilient member 232 is positioned on the external nasal dilator 200 in such a way that the lower resilient member 232 extends laterally across the lower section 228 from the left edge 218 to the right edge 220 and applies the second dilating force to the laterally opposed external nasal valves 116 when the external nasal dilator 200 is attached to the human nose. The lower resilient member 232 is distinct and separate from the upper resilient member 230.

In this first example, the longitudinal width of the lower resilient member 232 is larger than that of the upper resilient member 230. This is not always required yet may be advantageous to allow the lower resilient member 232 to apply the second dilating force in proximity to the external nasal valves 116, which are typically farther apart than the internal nasal valves 126.

Extending longitudinally between a bottom edge 234 of the upper resilient member 230 and a top edge 236 of the lower resilient member 232 and laterally between the left edge 218 and right edge 220 is an intermediate section 238. The left recess 221, right recess 223, and convergence points 222, 224 are within the intermediate section 238.

Referring specifically to FIG. 4, the back surface 206 forms a nose contacting surface that contacts the skin of the nose 100 and adheres the external nasal dilator 200 thereto using a skin contact adhesive 240. In certain examples, the skin contact adhesive 240 covers substantially the entire the back surface 206. In other examples, the skin contact adhesive 240 covers only portions of the back surface 206.

The skin contact adhesive 240 is an adhesive suitable for skin contact. Skin contact adhesives can adhere to the skin, but are removable without substantially damaging the skin. Examples of skin contact adhesives include, but are not limited to, acrylic adhesives, silicone adhesives, hydrogels, hydrocolloids, silicone, or the like. The adhesive material may also include an additive that provides benefits to the skin such as vitamins, vitamin E, and/or zinc oxide, for example.

In the example shown, a majority of the surface area of the back surface 206 is in the lower section 228, which places most of the adhesive surface area where the skin contact adhesive 240 is located on the lower section 228. This may be advantageous because at least some of the lower section 228 is intended to adhere to the ala 110 and, in some examples, the lower resilient member 232 applies a larger dilating force than the upper resilient member 230, which may require the additional adhesive surface are to mitigate peeling.

Referring to FIG. 6, the back surface 206 may be covered by a release liner 242 that forms part of the external nasal dilator's 200 packaging and is designed to cover the skin contact adhesive 240 prior to use. Before the wearer applies the external nasal dilator's 200 to the nose, the wearer removes the release liner 242 to expose the adhesive 240 and then adheres the external nasal dilator 200 to the nose.

The release liner 242 may have a single piece construction that covers the adhesive 240 or a two-piece construction with a seam 244 proximal to the midline M center of the external nasal dilator 200 as shown.

The release liner 242 is typically a paper or plastic film used to prevent the adhesive 240 from prematurely adhering. The release liner 242 may be composed of a base material that is coated on one or both sides with a release agent that facilitates easy removal from the adhesive 240. The release liner 242 may be made of any conventional nasal dilator release liner material, such as polymer-coated paper, for example.

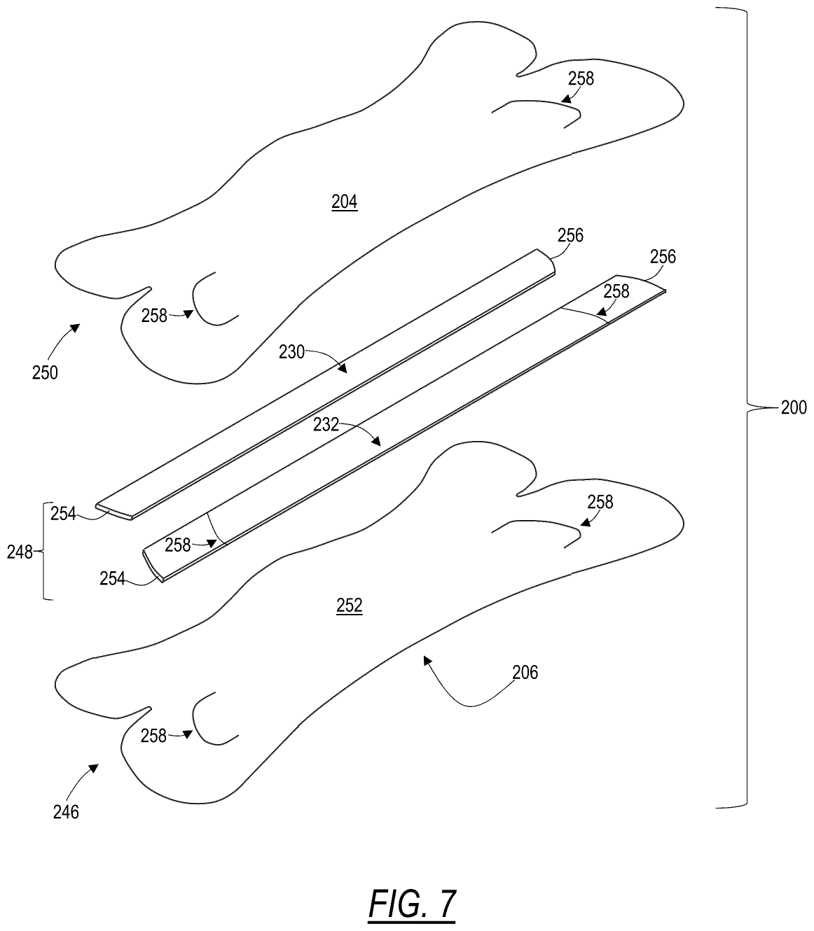

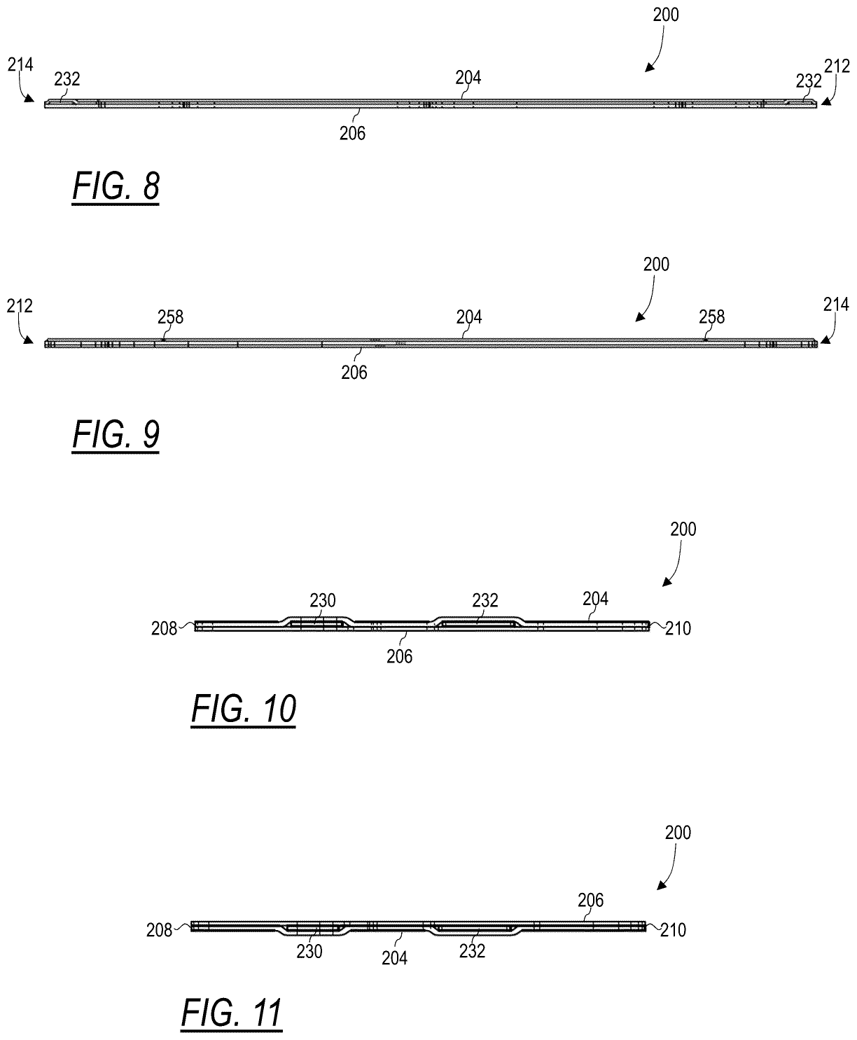

The external nasal dilator 200 is composed of a flexible substrate that carries the upper resilient member 230 and the lower resilient member 232. Referring to FIGS. 7-11, the flexible substrate may include multiple layers including a base layer 246, resilient layer 248, and cover layer 250 that are laminated together.

The base layer 246 and cover layer 250 may be composed of a thin, flexible material that is comfortable. Such a material may include, for example, woven or non-woven fabric, such as polyester, polyethylene, polypropylene, polyurethane, or the like. In the alternative, the material may include a plastic woven or non-woven material, such as polyethylene, polypropylene, polyurethane, or the like. In other examples, the material may be a foam, silicone, or the like.

The base layer 246 includes the back surface 206 having the skin contact adhesive 240 thereon for adhering the external nasal dilator 200 to the nose.

The base layer 246 includes a resilient layer engaging side 252 that engages with the resilient layer 248 such that the resilient layer 248 cooperatively mates with the base layer 246. The resilient layer engaging side 252 may include adhesive or the like that is capable of attaching the resilient layer 248 to the base layer 246.

In some particular examples, the base layer 246 has a thickness of about 0.5 to about 500 μm. The base layer 246 may be oxygen and/or moisture permeable or impermeable, depending on the materials chosen.

The resilient layer 248 includes the upper resilient member 230 and the lower resilient member 232. The upper resilient member 230 and lower resilient member 232 may extend completely to the left edge 218 and right edge 220 or may terminate proximal to, but short of, the left edge 218 and right edge 220.

In other examples, the resilient layer 248 may include a single resilient member or more than two resilient members. In the example shown, the upper resilient member 230 and lower resilient member 232 extend substantially parallel in the lateral direction of the external nasal dilator 200. The upper resilient member 230 and lower resilient member 232, respectively, include a first end 254 and a second end 256.

The upper resilient member 230 and lower resilient member 232 may be made from any suitable material having the desired flexibility and resiliency to generate the desired amount of dilating force, such as metal, plastic, or the like. The dilating force is the spring biasing force created by the resiliency of the resilient members 230, 232. The resilient members 230, 232 are typically flat, semi-rigid, and resilient so as to generate a dilating force when flexed across the nose, which is what lifts the exterior wall tissues of the nostrils. This dilating force is typically between about 5 grams and 35 grams, 15 and 35 grams, or sometimes about 25 grams. In a particular example, the resilient members 230, 232 are made of biaxially oriented polyester with a thickness of 0.1 mm to 0.3 mm and a longitudinal width of 3 mm to 6 mm. The longitudinal width and/or front to back thickness of the resilient members 230, 232 may be selected to obtain an external nasal dilator 200 with the desired amount of dilating force. Other particular examples of materials from which a resilient member 230, 232 can be made include polyethylene, polypropylene, or the like.

The dilating force strength of resilient members may be measured by conventional techniques. U.S. Pat. No. 11,344,444 provides such an example in which its resilient member is compressed into U-shape while a scale measures the force.

The resilient members 230, 232 may be secured to the resilient layer engaging side 252 of the base layer 246 by adhesive material thereon. The adhesive material may cover the resilient layer engaging side 252 or may be substantially the same size and shape as the resilient members 230, 232.

The number and design of the resilient members 230, 232 may depend on the desired amount of force to be applied to the nose, the direction of force to be applied to the nose, and aesthetic considerations.

The upper resilient member 230 and lower resilient member 232 are positioned between the base layer 246 and cover layer 250 and held in place by an adhesive, which also stops the base layer 246 and cover layer 250 from separating when the external nasal dilator 200 is being worn or when the external nasal dilator 200 is being removed.

The cover layer 250 is primarily made of a thin, flexible material. The cover layer 250 may be made of the same material as the base layer 246 or a different material from the base layer 246. The cover layer 250 and base layer 246 may be laminated together using adhesive, which may include a thermally fusable film. The cover layer 250 may have a thickness of about 1 to about 500 μm and may be oxygen and moisture permeable or impermeable. The cover layer 250 may also include a woven textile and/or nonwoven textile. The cover layer may also be colored to match different skin tones, or have decorative colors such as purple, for example. The cover layer 250 may also include decorative features and or indicia such as words, logos and the like printed thereon or attached thereto.

The cover layer 250 is optional in certain examples of the external nasal dilator 200.

In the example shown in FIGS. 3-11, the external nasal dilator 200 includes a pair of opposed cuts 258 that extend completely through the cover layer 250, resilient layer 248, and base layer 246. As shown in FIG. 7, the cuts 258 bisect the lower resilient member 232. The cuts 258 form a separation in in the lateral and longitudinal direction through the material making up each layer. The cuts 258 are positioned at laterally opposed ends of the lower resilient member 232. As shown in FIG. 6, the cuts 258 may extend through the release liner 242 as well.

In other examples, the cuts 258 do not extend completely through the cover layer 250, resilient layer 248, and base layer 246. In certain examples, the cuts extend through the upper resilient member 230 and/or lower resilient member 232 while not extending through at least one of the cover layer 250 or base layer 246.

The cuts 258 include an outside edge 260, an upper cut edge 262, and a lower cut edge 264. The outside edge 260 defines a longitudinal width of the cut 258. The upper cut edge 262 and a lower cut edge 264 extend laterally toward the midline M from the outside edge 260. The curvature of the outside edge 260 may mimic the portion of the left edge 218 or right edge 220 laterally adjacent to the cut 258.

The outside edge 260 separates the longitudinal width of the lower resilient member 232 and extends past the top edge 236 and bottom edge 266 thereof. The upper cut edge 262 and lower cut edge 264 extend inward toward the midline M substantially parallel to top edge 236 and bottom edge 266 of the lower resilient member 232.

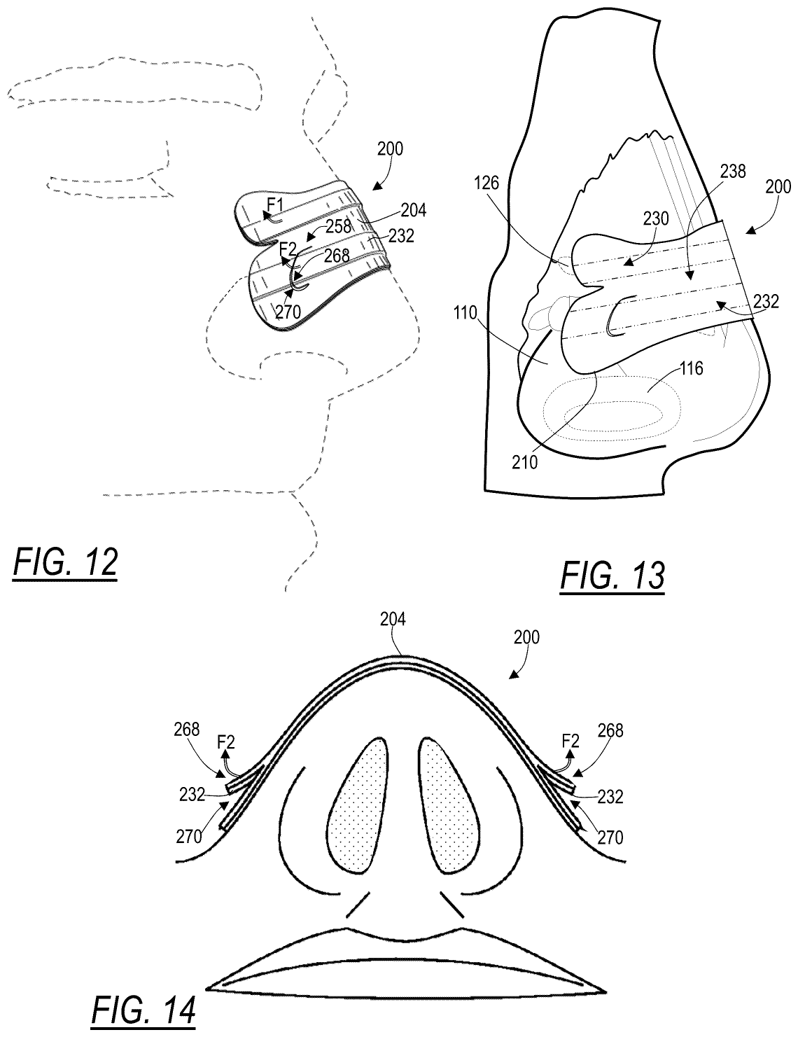

Referring to 12-14, the external nasal dilator 200 is shown adhered to the exterior wall of a human nose. When adhered to the nose, the external nasal dilator 200 applies dilating forces to the exterior wall, which lifts the exterior wall tissue of the nostrils and dilates the nasal cavities. Dilating the nasal cavities reduces airflow resistance and increases airflow through the nasal cavities when breathing. The right side of the nose is a mirror image of FIG. 12.

In FIG. 13, the external nasal dilator 200 is in its intended position on the nose relative to the positions of the internal nasal valves 126 and external nasal valves 116. The upper resilient member 230 is positioned proximal to and/or directly over the internal nasal valves 126 so that the first dilating force F1 is focused primarily on the internal nasal valves 126. The lower resilient member 232 is positioned at least partially on the ala 110 just above the external nasal valves 116 so that the second dilating force F2 is focused on the external nasal valves 116. The space in the intermediate section 238 between the upper resilient member 230 and lower resilient member 232 is sufficient to separate the first dilating force F1 and second dilating force F2 enough to focus these dilating forces at the desired location of the nose.

Also, as shown in FIG. 13, the lower section 228 is positioned on and/or slightly above the ala 110. This positioning places a majority of the adhesive surface area on the back surface 206 of the external nasal dilator 200 on or slightly above the ala 110 to assist with applying the second dilating force F2 to the ala 110 in close proximity to the external nasal valves 116. The concave curvature of the bottom edge 210 allows the bottom edge 210 to extend downwardly to just above the external nasal valves 116, which also permits for a large amount of adhesive surface area to be in close proximity to the external nasal valves 116, and beneath the lower resilient member 232. In this manner, a majority of the adhesive surface area on the back surface 206 of the external nasal dilator 200 is closer to the left edge 218 and right edge 220 than the midline M.

When adhered to and positioned across the bridge of the nose a material separation caused by the cuts 258 forms opposed raised flaps 268, leaving a gap 270 caused by the separation. The length of the flaps 268 determines the degree of separation. The flaps 268 redirect the angle of the dilating force F2 applied by the lower resilient member 232, changing it from primarily peel forces and tensile forces into primarily shear forces. This redirected dilating force is imparted to the external wall of the nose.

In other examples, the type, number, and location of cuts 258 may vary. In one particular example, there are a pair of opposed cuts 258 through the upper section 226 as well. In another example, there are a pair of opposed cuts 258 through the upper section 226 and not the lower section 228. In yet another example, the external nasal dilator 200 does not include any of the cuts 258.

The upper resilient member 230 and lower resilient member 232 each have an effective length responsible for creating the dilating force. In the first example, the effective length of the upper resilient member 230 is the lateral length from the first end 254 to the second end 256 thereof. The effective length of the lower resilient member 232 is less than that of the upper resilient member 230 because the cuts 258 define the effective length of the lower resilient member 232. The effective length of the lower resilient member 232 extends laterally between the opposed outside edges 260.

The cuts 258 reduce the amount of the lateral length of the lower resilient member 232 that creates the dilating force. This, in turn, allows the lower resilient member 232 to generate more dilating force than the lower resilient member 232 would otherwise be able to generate.

The upper resilient member 230 is laterally longer than the effective length of the lower resilient member 232. Because the upper resilient member is positioned on a skinnier part of the nose, the upper resilient member 230 may extend laterally beyond the nose. This may further help alleviate congestion.

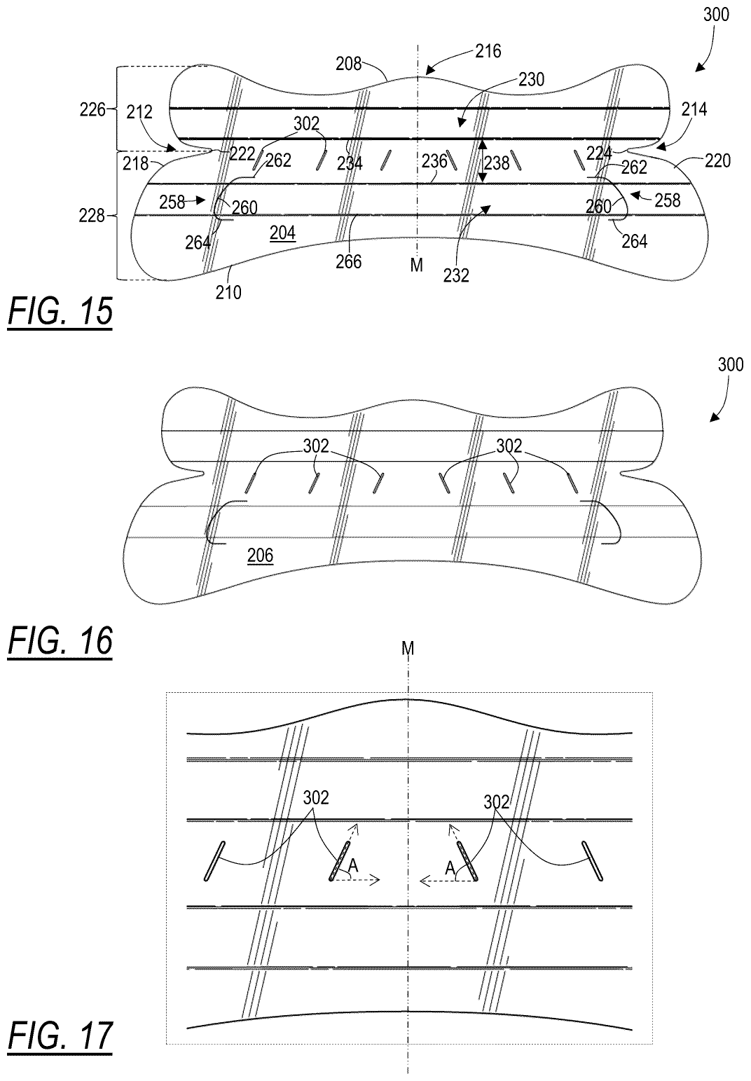

Another example of an external nasal dilator 300 will now be described by referring to FIGS. 15-17. The same reference numerals refer to features the second example has in common with the first example discussed above. This second example of the external nasal dilator 300 has the same general construction as the first example of the external nasal dilator 200, but for three features.

The external nasal dilator 300 is composed of substantially transparent materials such that the cover layer 250, resilient layer 248, and base layer 246 are substantially transparent. For the cover layer 250 and base layer 246 to be substantially transparent they may be made, for example, from a transparent plastic film. A plurality of slits 302 through the cover layer 250 and base layer 246 allow moisture to pass through the cover layer 250 and base layer 246 material.

The slits 302 are formed through the intermediate section 238 of the external nasal dilator 300 extending between the upper resilient member 230 and lower resilient member 232. The slits 302 are elongated in the longitudinal direction and are laterally spaced across the intermediate section 238. In the example shown, the slits 302 are longitudinally elongated towards the longitudinal midline M of the nose attachment member 304 at an angle A relative to the lateral direction.

In certain examples, the angle A is 15 degrees to 75 degrees, 18 degrees to 72 degrees to, 25 degrees to 65 degrees, or about 65 degrees.

In certain examples, the longitudinal length of the slits 302 is 2 mm to 4 mm, 2.5 mm to 3.5 mm, 2.8 mm, or about 3 mm.

In certain examples the lateral spacing between the slits 302 on the same side of the midline M is 5 mm to 10 mm, 6 mm to 8 mm, 6.5 mm to 7.5 mm, 6.8 mm, or about 7 mm.

In certain examples the lateral spacing between the adjacent slits 302 on either side the midline M is the same as above or different. In one particular example, this lateral spacing is 5 mm to 10 mm, 6 mm to 8 mm, 6 mm to 7 mm, or about 6.5 mm.

Angling the slits 302 towards the longitudinal midline M has at least two advantages. First, the angled slits 302 assist the wearer with aligning the nose attachment member on the nose. Second, angling the slits 302 as described reduces the risk of ripping the external nasal dilator 200 when removing it from the nose.

In other examples, the slits 302 may be positioned on other portions of the external nasal dilator 200 in combination with the intermediate section 238 or in the alternative to placing them in the intermediate section 238.

In the second example, the longitudinal width of the lower resilient member 232 is substantially equal to or closely approximating that of the upper resilient member 230. Using a lower resilient member 232 with a smaller longitudinal width may be desirable for certain people because such a lower resilient member 232 applies a smaller second dilating force F2 compared to the lower resilient member 232 in the first example.

It is to be understood that other examples of the external nasal dilator may include resilient members having relatively different longitudinal widths and/or lateral lengths than those shown in the drawings.

It should be understood that the slits 302 may be incorporated with other examples of the nasal dilator besides that shown in FIGS. 15-17, including the first example of the external nasal dilator 200.

Referring back to FIGS. 1, 2, 12, 13, and 14. when the external nasal dilator 200 is adhered to the exterior wall 108 of the nose 100, the upper resilient member 230 is positioned on the nose 100 in such a way that the upper resilient member 230 extends laterally across the bridge 104 of the nose 100 and applies the first dilating force F1 to the laterally opposed internal nasal valves 126. The lower resilient member 232 is positioned on the nose 100 in such a way that the lower resilient member 232 extends laterally across the bridge 104 of the nose 100 and applies the second dilating force F2 to laterally opposed external nasal valves 116 when the external nasal dilator 200 is attached to the human nose 100.

In certain examples, the second dilating force F2 may be greater than the first dilating force F1. This feature is sometimes advantageous because it may require more force to open the external nasal valves 116 than the internal nasal valves 126 due to the rigidity and thickness of the ala 100. In such cases, the second dilating force F2 may be, for example, 1.2, 1,5, 1.8, 2, 2.2, 2.4, 2.6, 2.8, or 3 times greater than the first dilating force F1.

Having the upper resilient member 230 and lower resilient member 232 distinct from one another may provide greater independence between the first dilating force F1 and second dilating force F2. The upper lobes 225a,b and the lower lobes 227a,b of the left end 212 and right end 214 proximal to the convergence points 222, 224 may provide additional independence between the first dilating force F1 and second dilating force F2. Together, these features allow the four corners of the external nasal dilator 200 to apply the dilating forces F1, F2 in four directions.

The convergence points 222, 224 are closer to the upper resilient member 230 than the lower resilient member 232. This feature is advantageous because it gives the lower section 228 more surface area for adhering to the lower part of the nose 100 and the larger ala 110. This, in turn, may help the lower section 228 remain adhered to the nose even though the second dilating force F2 is larger than the first dilating force F1. Having more surface area adhered to the ala 110 may help flare the external nasal valves 116.

The external nasal dilators 200, 300 described above may be used in a method of applying dilating force to a human nose. This method includes adhering the external nasal dilator 200, 300 to the exterior wall of the nose in such a way that the nose placement member 216 is positioned symmetrically about the bridge 104, the upper resilient member 230 is positioned on the exterior wall 108 proximal to the internal nasal valves 126, and the lower resilient member 232 is positioned on the exterior wall 108 proximal to the external nasal valves 116.

The external nasal dilator 200, 300 may be used in any situation in which the wearer desires to stabilize and or expand the nasal cavities 114, such as when ill, during exercise, while sleeping, and many other uses. Examples of methods that may be modified to manufacture the external nasal dilator are described in U.S. Pat. Nos. 8,062,329; 8,858,587; and 8,641,852.

The external nasal dilator 200, 300 may be manufactured by die cutting them from a continuous laminate of the material layers of FIG. 7. In another example, the individual components may be die cut individually from one or more continuous material layers before or during assembly of the completed laminate.

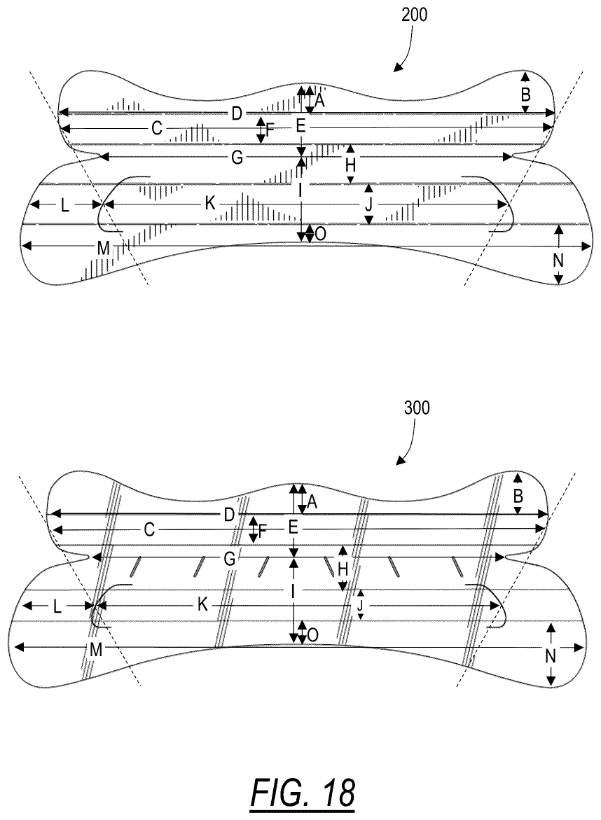

Referring to FIG. 18, measurements between certain points on the two examples of the nasal dilator shown in the drawings are marked with capital letters. The double-headed arrows indicate the points between which the measurements correspond. In Table 1, d1 represents the actual approximate measurements of the first example of the external nasal dilator 200, d2 represents the actual approximate measurements of the second example of the external nasal dilator 300. Ranges for each of these measurements are also provided by way of example only.

TABLE 1

| | d1 | d1 RANGE | d2 | d2 RANGE |
|---|---|---|---|---|
| LETTER | (mm) | (mm) | (mm) | (mm) |
| A | 3.0 | 2-6 | 3.0 | 2-6 |
| B | 4.6 | 2-7 | 4.6 | 2-7 |
| C | 53.3 | 45-65 | 53.3 | 45-65 |
| D | 53.8 | 45-65 | 53.8 | 45-65 |
| E | 7.8 | 6-9 | 7.8 | 6-9 |
| F | 3.2 | 2-4 | 3.2 | 2-4 |
| G | 44 | 35-50 | 44 | 35-50 |
| H | 4.5 | 3.5-5.5 | 4.5 | 3.5-5.5 |
| I | 9.4 | 8-11 | 9.4 | 8-11 |
| J | 4.5 | 3.5-5.5 | 3.2 | 2-4 |
| K | 43.9 | 40-48 | 43.9 | 40-48 |
| L | 8.1 | 6-10 | 8.1 | 6-10 |
| M | 62.7 | 55-75 | 62.7 | 55-75 |
| N | 6.6 | 3-10 | 7.4 | 4-10 |
| O | 2 | 1-3 | 2.5 | 1.5-3.5 |

In FIG. 18, the dashed lines connect the end of the upper resilient member 230 with the cuts 258. This illustrates the difference in the effective length of the upper resilient member 230 and lower resilient member 232, showing the effective length of the lower resilient member 232 to be shorter than the effective length of the upper resilient member 230.

The measurements of Table 1, correspond with relative dimensions of different portions of the external nasal dilator 200,300. Dividing one of the measurements by another, provides a ratio between the measurements. These ratios form part of the disclosure.

Multiple external nasal dilators may be packaged for sale by placing each dilator in an individual wrapper and placing the wrapped dilators into a box, bag, or the like for consumers to purchase. Packages of multiple dilators may contain dilators of the same dilating force strength or dilators of different recoil strengths. In some examples, the wrappers may form an array of dilators having different dilating force strengths. In other examples, the wrappers and/or dilators themselves may include a color or other indicia thereon that indicates the relative dilating force strength of the external nasal dilator.

The external nasal dilator and related methods are not limited to the details and features described in connection with the example embodiments. There are numerous variations and modifications of the nasal dilator and methods that may be made without departing from the scope of what is claimed.

That which is claimed is:

1. An external nasal dilator configured to dilate laterally opposed internal nasal valves and external nasal valves of a human nose, the external nasal dilator comprising:
a flexible substrate having an adhesive back surface for adhering the external nasal dilator to a human nose, the flexible substrate having a top edge, bottom edge, a left edge, and a right edge collectively defining an outer perimeter of the external nasal dilator, the left edge and the right edge converging inwardly toward each other to form a left recess along the left edge and a right recess along the right edge, the left recess and right recess dividing the flexible substrate into two laterally opposed upper lobes and two laterally opposed lower lobes;
an upper section of the flexible substrate including the two laterally opposed upper lobes and carrying an upper resilient member extending laterally from one upper lobe to the other upper lobe; wherein, when the external nasal dilator is adhered to the nose, the upper resilient member applies a first dilating force to the internal nasal valves;
a lower section of the flexible substrate including the two laterally opposed lower lobes and carrying a lower resilient member that is distinct from the upper resilient member and extends laterally from one lower lobe to the other lower lobe, the lower resilient member and the lower section being laterally elongated relative to the upper resilient member and the upper section, respectively; wherein, when the external nasal dilator is adhered to the nose, the lower resilient member applies a second dilating force to the external nasal valves; and
an intermediate section of the flexible substrate extending laterally between and longitudinally separating the upper resilient member and the lower resilient member, the left recess and the right recess being in the intermediate section.

2. The external nasal dilator of claim 1, wherein the intermediate section, right recess, and left recess separate the first dilation force at the upper lobes from the second dilation force at the lower lobes.

3. The external nasal dilator of claim 1, wherein the adhesive back surface has greater surface area on the lower section than the upper section.

4. The external nasal dilator of claim 1, wherein the second dilation force is greater than the first dilation force.

5. The external nasal dilator of claim 1, wherein a left convergence point defining an innermost edge of the left recess and a right convergence point defining an innermost edge of the right recess are closer to the upper resilient member than the lower resilient member.

6. The external nasal dilator of claim 1, further comprising a plurality of discrete slits through the intermediate section, the discrete slits being laterally spaced apart and longitudinally elongated.

7. The external nasal dilator of claim 1, further comprising:
a pair of opposed cuts extending longitudinally through the flexible substrate, respectively, at laterally opposed ends of the lower resilient member, the opposed cuts defining a pair of opposed flaps of the lower resilient member that direct the second dilating force; and
wherein the upper resilient member has a lateral length from a left end thereof to a right end thereof and a distance between the opposed cuts is less than the lateral length.

8. An external nasal dilator configured to dilate laterally opposed internal nasal valves and external nasal valves of a human nose, the external nasal dilator comprising:
a flexible substrate having an adhesive back surface for adhering the external nasal dilator to a human nose, the flexible substrate having a top edge, bottom edge, a left edge, and a right edge collectively defining an outer perimeter of the external nasal dilator;
an upper section of the flexible substrate carrying an upper resilient member extending laterally across the upper section; wherein, when the external nasal dilator is adhered to the nose, the upper resilient member applies a first dilating force to the internal nasal valves;
a lower section of the flexible substrate carrying a lower resilient member that is distinct from the upper resilient member and extends laterally across the lower section; wherein, when the external nasal dilator is adhered to the nose, the lower resilient member applies a second dilating force to the external nasal valves;
an intermediate section of the flexible substrate extending laterally between, and longitudinally separating, the upper resilient member and the lower resilient member; and
a plurality of discrete slits through the intermediate section, the discrete slits being laterally spaced apart and longitudinally elongated.

9. The external nasal dilator of claim 8, wherein:
the left edge and the right edge converge inwardly toward each other to form a left recess along the left edge and a right recess along the right edge, the left recess and right recess dividing the flexible substrate into two laterally opposed upper lobes and two laterally opposed lower lobes;
the upper section includes the two laterally opposed upper lobes and the upper resilient member extends laterally from one upper lobe to the other upper lobe;
the lower section includes the two laterally opposed lower lobes and the lower resilient member extends laterally from one lower lobe to the other lower lobe;
the lower resilient member and the lower section are laterally elongated relative to the upper resilient member and the upper section, respectively; and
the left recess and the right recess are in the intermediate section.

10. The external nasal dilator of claim 8, wherein the slits are longitudinally elongated towards a longitudinal midline of the external nasal dilator.

11. The nasal dilator of claim 8, wherein the slits are longitudinally elongated towards a longitudinal midline of the external nasal dilator at an angle of 15° to 75° relative to the lateral direction.

12. The external nasal dilator of claim 8, wherein:
the left edge and the right edge converge inwardly toward each other to form a left recess along the left edge and a right recess along the right edge, the left recess and right recess dividing the flexible substrate into two laterally opposed upper lobes and two laterally opposed lower lobes;
the upper section includes the two laterally opposed upper lobes and the upper resilient member extends laterally from one upper lobe to the other upper lobe;
the lower section includes the two laterally opposed lower lobes and the lower resilient member extends laterally from one lower lobe to the other lower lobe;
the left recess and the right recess are in the intermediate section; and
the intermediate section, right recess, and left recess separate the first dilation force at the upper lobes from the second dilation force at the lower lobes.

13. The external nasal dilator of claim 8, wherein the adhesive back surface has greater surface area on the lower section than the upper section.

14. The external nasal dilator of claim 8, wherein the lower resilient member and the lower section are laterally elongated relative to the upper resilient member and the upper section.

15. The external nasal dilator of claim 8, wherein:
the adhesive back surface has greater surface area on the lower section than the upper section;
the lower resilient member and the lower section are laterally elongated relative to the upper resilient member and the upper section; and
the second dilation force is greater than the first dilation force.

16. The external nasal dilator of claim 8, wherein:
the left edge and the right edge converge inwardly toward each other to form a left recess along the left edge and a right recess along the right edge, the left recess and right recess dividing the flexible substrate into two laterally opposed upper lobes and two laterally opposed lower lobes;
the upper section includes the two laterally opposed upper lobes and the upper resilient member extends laterally from one upper lobe to the other upper lobe;
the lower section includes the two laterally opposed lower lobes and the lower resilient member extends laterally from one lower lobe to the other lower lobe;
the left recess and the right recess are in the intermediate section; and
a left convergence point defining an innermost edge of the left recess and a right convergence point defining an innermost edge of the right recess are closer to the upper resilient member than the lower resilient member.

17. The external nasal dilator of claim 8, further comprising:
a pair of opposed cuts extending longitudinally through the flexible substrate at respective laterally opposed ends of the lower resilient member, the opposed cuts defining a pair of opposed flaps of the lower resilient member that direct the second dilating force; and
wherein the upper resilient member has a lateral length from a left terminal end thereof to a right terminal end thereof and a distance between the opposed cuts is less than the lateral length.

18. The external nasal dilator of claim 8, further comprising:
a pair of opposed cuts extending longitudinally through the flexible substrate, respectively, at laterally opposed ends of the lower resilient member, the opposed cuts defining a pair of opposed flaps of the lower resilient member that direct the second dilating force; and
wherein the upper resilient member has a lateral length from a left terminal end thereof to a right terminal end thereof and a distance between the opposed cuts is less than the lateral length.

19. An external nasal dilator configured to dilate laterally opposed internal nasal valves and external nasal valves of a human nose, the external nasal dilator comprising:
a flexible substrate having an adhesive back surface for adhering the external nasal dilator to a human nose, the flexible substrate having a top edge, bottom edge, a left edge, and a right edge collectively defining an outer perimeter of the external nasal dilator, the left edge and the right edge converging inwardly toward each other to form a left recess along the left edge and a right recess along the right edge, the left recess and right recess dividing the flexible substrate into two laterally opposed upper lobes and two laterally opposed lower lobes;
an upper section of the flexible substrate including the two laterally opposed upper lobes and carrying an upper resilient member extending laterally from one upper lobe to the other upper lobe; wherein, when the external nasal dilator is adhered to the nose, the upper resilient member applies a first dilating force to the internal nasal valves;
a lower section of the flexible substrate including the two laterally opposed lower lobes and carrying a lower resilient member that is distinct from the upper resilient member and extends laterally from one lower lobe to the other lower lobe; wherein, when the external nasal dilator is adhered to the nose, the lower resilient member applies a second dilating force to the external nasal valves;
an intermediate section of the flexible substrate extending laterally between, and longitudinally separating, the upper resilient member and the lower resilient member, the left recess and the right recess being in the intermediate section; and
a left convergence point defining an innermost edge of the left recess and a right convergence point defining an innermost edge of the right recess, the left convergence point and the right convergence point being closer to the upper resilient member than the lower resilient member.

20. The external nasal dilator of claim 19, wherein the intermediate section, right recess, and left recess separate the first dilation force at the upper lobes from the second dilation force at the lower lobes.

21. The external nasal dilator of claim 19, wherein the adhesive back surface has greater surface area on the lower section than the upper section.

22. The external nasal dilator of claim 19, wherein the second dilation force is greater than the first dilation force.

23. The external nasal dilator of claim 19, further comprising a plurality of discrete slits through the intermediate section, the discrete slits being laterally spaced apart and longitudinally elongated.

24. The external nasal dilator of claim 19, further comprising:
a pair of opposed cuts extending longitudinally through the flexible substrate, respectively, at laterally opposed ends of the lower resilient member, the opposed cuts defining a pair of opposed flaps of the lower resilient member that direct the second dilating force; and
wherein the upper resilient member has a lateral length from a left end thereof to a right end thereof and a distance between the opposed cuts is less than the lateral length.
